(19) United States
(12) Patent Application Publication
(10) Pub. No.: US 2007/0243182 A1
(43) Pub. Date: Oct. 18, 2007

(54) Use of Carboxypeptidease G for Combating Antifolate Toxicity

(75) Inventors: Roger Melton, Wiltshire (GB); Anthony Atkinson, Wiltshire (GB)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Protherics Medicines Development Limited, Runcorn, Cheshire (GB)

(21) Appl. No.: 10/590,789

(22) PCT Filed: Feb. 28, 2005
(86) PCT No.: PCT/GB05/00751

§ 371 Date: Feb. 12, 2007

(30) Foreign Application Priority Data
Feb. 28, 2004 (GB) 0404487.1

(51) Int. Cl. A61K 38/48; C12N 9/14; C12Q 1/34

(52) U.S. Cl. 424/94.63; 435/195; 435/24

(57) ABSTRACT

A method of combating toxicity caused by an antifolate compound of Formula I, 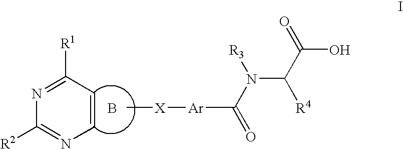 in an individual who has been administered the compound. The method comprises administering an enzyme that has carboxypeptidase G activity to the individual.

The present invention relates to the uses of an enzyme having carboxypeptidase G activity, and in particular to its use in combating toxicity caused by antifolate compounds.

Natural folates are used by cells in the folate pathway to synthesise DNA, RNA and in protein synthesis, and are therefore essential dietary requirements (Jolivet et al, 1983; Pinedo et al, 1976; Goldman 1975).

The three enzymes in the folate pathway most studied as targets for antifolate drugs are dihydrofolate reductase (DHFR), thymidylate synthase (TS) and glycinamide ribonucleotide formyltransferase (GARFT). DHFR and TS, along with serine hydroxymethyltransferase (SHMT), comprise the three enzymes of the thymidylate cycle. SHMT catalyzes the conversion of serine to glycine with the formation of methylenetetrahydrofolic acid (MTHF). MTHF, under the influence of TS, donates its methylene group to deoxyuridylic acid to form thymidylate, an essential component of DNA. Importantly, in the TS reaction, tetrahydrofolate (THF) supplies reducing equivalents for the conversion of the methylene group of MTHF to the methyl group of thymidylate (dTMP). Thus, for every molecule of dTMP formed, a molecule of THF is converted to dihydrofolate (DHF). DHF must be converted back to THF so that TS cycle should continue producing dTMP. This reaction is catalyzed by DHFR which utilizes NADPH as the reductant. DHFR also catalyzes the conversion of folic acid to DHF.

GARFT catalyzes the third in the series of ten reactions required for de novo purine biosynthesis, the conversion of glycinamide ribonucleotide to formylglycinamide ribonucleotide utilizing 10-formylTHF as the formyl donor. GARFT occurs in manuals as a trifunctional protein which catalyzes the second and the fifth steps on this pathway in addition to the third. GARFT activity resides in the carboxy-terminal portion of this tri-functional protein. De novo purine biosynthesis leads to the formation of inosine monophosphate, the precursor of ATP and GTP necessary for RNA formation and of dATP and dGTP necessary for DNA formation.

Inhibition of DHFR leads to a deficiency of dTMP because DHF cannot be recycled for use in the TS reaction. This in turn leads to deficient DNA synthesis, DNA breakdown and cell death. Direct inhibition of TS likewise leads to a deficiency of dTMP and cell death. Direct inhibition of GARFT leads to depletion of purine nucleotides, which also leads to cell death, but the degree of cell kill is generally less than that produced by an equally growth-inhibitory concentration of a TS inhibitor (Kisliuk et al, 2003).

Methotrexate (MTX), a synthetic folate analogue, has been in clinical use since 1948 (Bleyer 1978) and is an important component of various chemotherapeutic regimens used for the treatment of patients with neoplastic diseases. The cytotoxic effects of both MTX and its active metabolites is through the inhibition of DHFR leading to inhibition of DNA synthesis, repair and cellular replication. Actively proliferating tissue, such as malignant cells, are in general more sensitive to this cellular interference by MTX. In addition, MTX has immunomodulating effects and is used in the treatment of a number of other diseases, such as rheumatoid arthritis (RA), multiple sclerosis (MS) and psoriasis. Application of high doses of MTX, usually administered as a prolonged infusion, is nowadays frequently used in patients with Non-Hodgkin's Lymphoma (NHL), acute lymphoblastic leukemia (ALL) or soft tissue tumours such as osteosarcoma.

Although actively proliferating malignant tissues are most sensitive to MTX, MTX can still be toxic to healthy cells in a dose and time dependent manner through two principle mechanisms. The first is common to all antifolates. This mechanism is through the inhibition of DNA synthesis and cellular metabolism, which is the underlying mechanism that is responsible for MTX's cytotoxic anti-cancer action. The risk of significant toxicity to healthy cells correlates with increasing doses of MTX and the time of exposure. MTX therapy is associated with a spectrum of toxicities, with myelosuppression, mucositis, acute hepatitis and nephrotoxicity being the most frequent and serious complications (Bleyer 1978). Additional toxicities seen with high dose therapy are acute desquamative dermatitis, B-lymphocyte dysfunction, and neurological effects. Similar common toxicities are also caused by other antifolate drugs, although they have generally been administered at lower doses than MTX.

The second mechanism is MTX induced renal tubular obstruction and consequent renal dysfunction (MTX nephrotoxicity). MTX is metabolised by liver aldehyde oxidase to 7-hydroxy-MTX. The aqueous solubility of 7-hydroxy-MTX is 3 to 5 times lower than that of the parent compound and, under certain conditions, is known to precipitate in the renal tubules which is thought to be a principal mechanism in the pathogenesis of the MTX nephrotoxicity (Kintzel 2001, Condit 1969). Normal kidney function will accommodate removal of a particular load in a given time, thereafter accumulation and damage will ensue. If a patient receiving MTX develops nephrotoxicity leading to impaired elimination of MTX, a self-perpetuating cycle is initiated of reduced elimination, sustained high plasma MTX levels and subsequent exacerbation of both non-renal toxicity and progression of renal tubular damage, eventually leading to the death of the patient (although mortality can occur even in the absence of total renal failure).

Renal toxicity has been recorded with other antifolate compounds, but this may not be due to an analogous 7-hydroxylation of the compound. 7-OH-MTX toxicity occurs at high MTX doses, while the typical administered doses of the further antifolates described herein would only be considered to be ‘intermediate’ doses for MTX.

As a result of fatal outcomes from MTX toxicity, protection measures are routinely included in MTX therapeutic regimes:

1. Leucovorin rescue: Leucovorin calcium is the calcium salt of 5-formyl tetrahydrofolic acid (also known as folinic acid/folinate), the DHFR metabolite of folic acid and an essential coenzyme for nucleic acid synthesis and is not inhibited by MTX (Immunex Corporation 2001). As a result, leucovorin calcium is able to rescue MTX inhibited cells. However, at high MTX concentrations, leucovorin calcium may fail to prevent systemic toxicities (Goldman, 1975; Pinedo, 1976). Reversal of MTX by leucovorin calcium is competitive, with relatively higher concentrations required as the MTX concentration increases. When concentrations of MTX reach 100 μM, even ten-fold higher leucovorin calcium concentrations (1,000 μM) are unable to protect bone marrow cells from toxicity (Pinedo, 1976).

2. Hydration and alkalinisation is required to enhance the solubility of MTX and thus prevent MTX nephrotoxicity, which can lead to renal impairment.

With these measures, the incidence of life-threatening MTX toxicity may be lowered to around 1.5%. However, despite these precautions, prolonged MTX-clearance due to drug-related renal insufficiency may develop and lead to severe and life-threatening systemic toxicities, such as myelosuppression, mucositis, hepatitis and dermatitis. In the past, several attempts have been made to ameliorate systemic MTX toxicity in such patients. First, haemo- or peritoneal dialysis may enhance MTX clearance but usually result in only small and transient decreases of toxic serum MTX levels. Second, administration of thymidine or an intensified leucovorin rescue may lower systemic MTX toxicity but does not enhance MTX excretion.

MTX remains, with the limited exceptions, the only antifolate anticancer agent in clinical use to this date. Because of the relative safety and utility of MTX, considerable effort has been invested in attempting to design more therapeutically selective antifolates or antifolates with a wider tumor spectrum. Initially, the design was based on the burgeoning knowledge of folate-dependent pathways and the determinants of the mechanism of action of MTX. These determinants include transport, the tight-binding inhibition of its target, DHFR, and metabolism of MTX to poly-γ-glutamate (Gluₙ) metabolites. These early studies led to the development of other antifolate inhibitors of two types: (1) “classical” analogs that use the same cellular transport systems as MTX and are also metabolized to Gluₙ; and (2) “nonclassical” (i.e., lipophilic) analogs that do not require transport systems and that are not metabolized to Gluₙ. Although several of these analogs have undergone clinical trial, none has yet proved superior to MTX (McGuire, 2003).

Detailed examination of the mechanisms of cytotoxicity and selectivity of MTX showed that inhibition of both dTMP synthesis and de novo purine synthesis, secondary to DHFR inhibition, led to DNA synthesis inhibition and subsequent cell death; inhibition of other folate-dependent pathways did not appear necessary for cell death. Further studies showed that the contribution of inhibition of dTMP or purine synthesis to cell death varied in different cell types. These data suggested that inhibition of one of these pathways individually might (at least in some cases) be therapeutically superior to the dual inhibition induced by MTX. Thus in rational design and in structure-based design studies, two new classes of antifolate enzyme inhibitors were elaborated: direct inhibitors of TS and direct inhibitors of one or both of the two folate-dependent enzymes of de novo purine synthesis. Members of each class included both classical and nonclassical types. After preclinical evaluation, several of these have moved into clinical trials. To date only two new antifolate compounds have been approved for routine use; Tomudex (D1694, raltitrexed) is currently approved in Europe for the treatment of colon cancer and Pemetrexed (Alimta) has been approved in the US for pleural mesothelioma. This still represents a step forward for antifolates since, for example, MTX is ineffective against colon cancer.

Antifolate development continues. Based on the immense body of knowledge now extant on antifolates, specific aspects of the mechanism of action have been the focus. Newer antifolates have been described that inhibit more than one pathway in folate metabolism, that have improved delivery, or that inhibit other targets in folate metabolism. These new analogs are in various stages of preclinical and clinical development (McGuire, 2003; KisIiuk, 2003; Purcell & Ettinger, 2003; each of which is incorporated by reference in this entirety).

As with MTX, one major drawback to the clinical use of the newer antifolate drugs is an unacceptable level of toxicity. The ability to degrade these antifolate drugs rapidly in vivo would have two major clinical advantages. Firstly, it would minimize toxicity caused by the antifolate drugs. It also allows a higher dose of the antifolate compounds to be administered, potentially leading to a greater clinical effect. The lower toxicity and higher efficacy may be sufficient to realise the clinical promise of a number of drugs that have not progressed through clinical trials. Furthermore, as toxicity associated with antifolate drugs is normally duration-related rather than dose-related, the ability to rapidly remove excess free drug at a given time point may be therapeutically very useful.

Carboxypeptidase G₂ (CPG₂) is a folate-degrading enzyme from Pseudomonas sp. strain RS-16 (now reclassified as Variovorax paradoxus) and is a zinc-dependent dimeric protein of 83,000-84,000 Dalton (Kalghati & Bertino, 1981; Chabner et al, 1972; McCullough et al, 1971; Minton et al, 1983; and Sherwood et al, 1985). CPG₂ has a relatively restricted specificity and hydrolyses the C-terminal glutaric acid residue of folic acid, poly-glutamyl derivatives of folic acid, folate analogues, e.g. methotrexate, and sub-fragments of folic acid e.g., p-aminobenzoyl glutamate (Minton et al, 1983). To date carboxypeptidase enzymes have only been characterised in a small number of Pseudomonas sp. and can be separated on the basis of their substrate affinities for folate and its analogues (Kalghatgi and Bertino, 1981).

Sherwood et al (1985) have previously reported that CPG₂ follows Michaelis-Menten kinetics with Kₘ values of 4 μM for folate, 8 μM for MTX, 34 μM for 5-methyl THF, and 120 μM for 5-formyl TIE (leucovorin). CPG₂ cleaves methotrexate (MTX) into inactive metabolites, 4-deoxy-4-amino-N¹⁰-methylpteroic acid (DAMPA) and glutamate, and thus may provide an alternative route of MTX elimination particularly in patients who develop renal dysfunction due to MTX nephrotoxicity (Adamson et al, 1991; Mohty et al, 2000; von Poblozki et al, 2000; Widemann et al, 2000).

Para-aminobenzoyl glutamate is a substrate of CPG₂ as are a number of mustard prodrugs based on p-aminobenzoyl glutamate (Springer et al (1995); Dowell et al (1996)). However, there have been no reported studies that have attempted to assess whether any of the new antifolate drugs are substrates for CPG₂ cleavage. Prior to the present invention, it was not known whether any folate compounds other than folic acid, MTX, 5-methyl THF, and 5-formyl THF were substrates for CPG₂. It was not known, and could not predicted, whether any of the new generation of antifolate drugs are substrates for CPG₂ cleavage.

We have now shown that Tomudex (raltitrexed) is a substrate for CPG₂ and has a Kₘ of 7.8 μM and a k_{cat} of 24/s as measured by spectrophotometric assay. All the known substrates for CPG₂ have a benzene ring at a position equivalent to the five-membered ring containing A⁶ in Formula I, see below. Furthermore, the mustard prodrug p-aminobenzoyl glutamate derivatives assessed by Springer et al (1995) suggest that modifications on this benzene ring may be detrimental to carboxypeptidase activity. Tomudex, by contrast, has a thiophene ring at this position which is not an isosteric replacement for the benzene ring. Thus Tomudex, along with other antifolates with a thiophene at this position such as the GARFT inhibitors LY309987, AG2034 and AG2037 (McGuire, 2003) would not have been predicted to be substrates of CPG₂. Similarly, the furan-based equivalents would also not have been predicted to be substrates of CPG₂.

Antifolate compounds are useful in treating a range of medical conditions, particularly cancers, and being able to combat toxicity associated with these compounds will significantly increase their therapeutic value.

A first aspect of the invention provides a method of combating toxicity caused by an antifolate compound of Formula I, 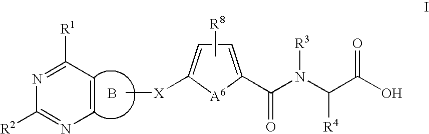
wherein
R¹ represents NH₂, OH or CH₃;
R² represents NH₂ or C₁₋₄ alkyl;
the group B represents a structural fragment of Formula Ia, Ib, Ic, Id or Ie, 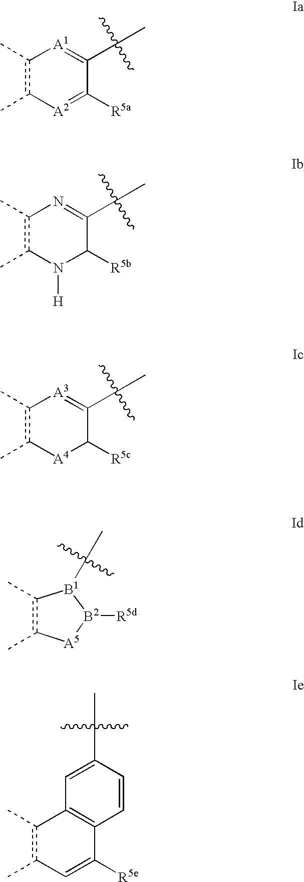
in which groups the dashed lines indicate the point of ring fusion with the pyrimidinyl ring and the wavy lines indicate the point of attachment of the structural fragments to the group X;
R^{5a} to R^{5e} independently represent H or C₁₋₄ alkyl;
A¹ represents C(R^{6a}) or N;
A² represents CH or N;
A³ represents C(H)R^{6b}, NR^{6c} or S;
A⁴ and A⁵ independently represent CH₂, NH, O or S;
the group B¹-B² represents CH—CH or C═C;
R^{6a} to R^{6c} independently represent H or C₁₋₄ alkyl, or R^{6c} represents C(O)R^{6d}, or
R^{6c}, together with R^{7b} represents C₁₋₂ n-alkylene;
R^{6d} represents H or C₁₋₄ alkyl;
X represents —CH₂C(H)R^{7a}— or —CH₂NR^{7b}— (in which latter two groups the CH₂ moiety is attached to the fused, pyrimidine-based heterocyclic group);
R^{7a} and R^{7b} independently represent H, C₁₋₆ alkyl, C₃₋₆ alkenyl or C₃₋₆ alkynyl, or R^{7b}, together with R^{6c} represents C₁₋₂ n-alkylene;
A⁶ represents O or S;
R⁸ represents H or one or two substituents selected from halo, C₁₋₄ alkyl and C₁₋₄ alkoxy;
R³ represents H or C₁₋₄ allyl;
R⁴ represents —CH₂C(R^{9a})(R^{9b})-D;
R^{9a} and R^{9b} independently represent H or C₁₋₄ alkyl, or R^{9a} and R^{9b} together represent ═C(H)R¹⁰;
R¹⁰ represents H or C₁₋₄ alkyl;
D represents C(O)OH, tetrazol-5-yl, (CH₂)₀₋₁—NHR¹¹, or, when R^{9a} and R^{9b} together represent ═C(H)R¹⁰, then D may also represent H, or D represents a structural fragment of Formula IIIa or IIIb, 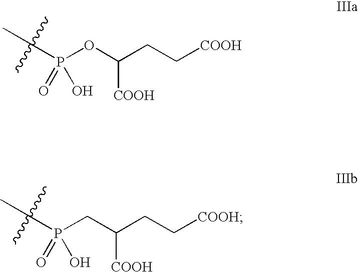
wherein the wavy lines indicate the point of attachment of the structural fragments;
R¹¹ represents H or C(O)R¹²;
R¹² represents H or phenyl substituted by C(O)OH and optionally substituted by one or two further substituents selected from halo, C₁₋₄ alkyl and C₁₋₄ alkoxy; and
alkyl, alkenyl and alkynyl groups, as well as the alkyl part of alkoxy groups, may be substituted by one or more halo atoms;
or a pharmaceutically acceptable salt and/or solvate thereof,
in an individual who has been administered said compound, the method comprising administering to the individual an enzyme that has carboxypeptidase G activity.

The term “halo”, when used herein, includes fluoro, chloro, bromo and iodo.

Pharmaceutically acceptable salts of compounds of Formula I (as well as of compounds comprising a structural fragment of Formula VIII described below) that may be mentioned include both acid addition salts and metal (e.g. alkali metal, such as sodium or potassium) salts. Solvates that may be mentioned include hydrates.

Compounds of Formula I may exhibit tautomerism. In particular, compounds of Formula I in which R¹ represents OH may alternatively be depicted as follows.

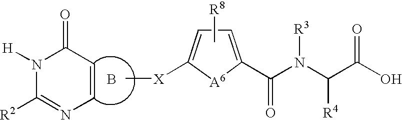

All tautomeric forms and mixtures thereof are included within the scope of the invention.

Compounds of Formula I that may be mentioned include the following.

(1). Compounds of Formula I in which X represents —CH₂NR^{7b}— and B represents a structural fragment of Formula Ia in which A¹ and A² both represent N or a structural fragment of Formula Ic.

(2) Compounds of Formula I in which X represents —CH₂NR^{7b}—.

(3) Compounds of Formula I in which X represents —CH₂C(H)R^{7a}—.

(4) Compounds of Formula I in which D represents tetrazol-5-yl, (CH₂)₀₋₁—NHR¹¹, or, when R^{9a} and R^{9b} together represent ═C(H)R¹⁰, then D may also represent H, or D represents a structural fragment of Formula IIIa or IIIb.

(5) Compounds of Formula I in which R^{6b}, together with R^{7b}, represents C₁₋₂ n-alkylene and the group B represents a structural fragment, of Formula Ic, Id or Ie, or a structural fragment of Formula Ia in which A¹ and/or A² represents CH.

(6) Compounds of Formula I in which the group B represents a structural fragment of Formula Id or Ie, or a structural fragment of Formula Ia in which A¹ and A² both represent CH.

(7) Compounds of Formula I in which R¹ represents OH and R^{7b} represents C₁₋₆ alkyl, C₃₋₆ alkenyl or C₃₋₆ alkynyl, or R^{7b}, together with R^{6c} represents C₁₋₂ n-alkylene.

Other compounds of Formula I that may be mentioned include those in which:

R¹ represents NH₂ or, particularly, OH;

R² represents NH₂ or methyl;

the group B represents a structural fragment of Formula Ia, Ic or Id;

A¹ and A² both represent N or, particularly, both represent CH;

R^{5a}, R^{5c} and R^{5d} independently represent H or, when A¹ and A² both represent CH,

R^{5a} may also represent methyl;

A³ represents S or, particularly, CH₂;

A⁴ and A⁵ independently represent NH;

the group B¹-B² represents CH—CH;

R^{7a} represents H, methyl or ethyl;

R^{7b} represents methyl or —CH₂C≡CH;

A⁶ represents S;

R⁸ represents methyl or, particularly, H;

R³ represents H;

R^{9a} and R^{9b} both represent H or R^{9a} and R^{9b} together represent ═CH₂;

R¹¹ represents phenyl ortho-substituted by C(O)OH.

Still further compounds of Formula I that may be mentioned include those in which:

R² represents methyl;

the group B represents a structural fragment of Formula Ia;

R^{5a} represents H;

X represents —CH₂NR^{7b}—;

R^{7b} represents methyl;

D represents C(O)OH;

R^{9a} and R^{9b} both represent H.

In a preferred embodiment, the compound of Formula I is one in which A⁶ represents S (i.e. the compound comprises a 2,5-thienyl group). Suitable compounds include Tomudex (Formula IV), LY309987 (Formula V), AG2034 (Formula VI) and AG2037 (Formula VII). Tomudex is most preferred.

In another embodiment, the compound of Formula I is one in which X represents —CH₂NR^{7b}— wherein R^{7b} represents —CH₂C≡CH.

By “an enzyme that has carboxypeptidase G activity” we include the meaning of an enzyme that hydrolyses the C-terminal L-glutamic acid residue from folic acid, folate analogues, and sub-fragments of folic acid e.g., p-aminobenzoyl glutamate.

Preferably, the enzyme that has carboxypeptidase G activity is carboxypeptidase G₂ (CPG₂), EC number 3.4.22.12.

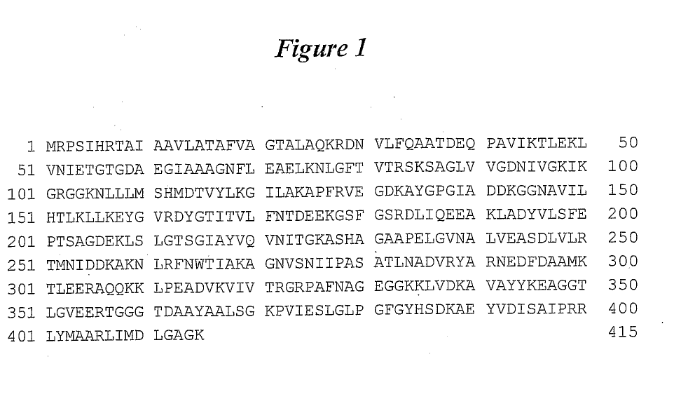

The sequence of the gene encoding CPG₂ and the CPG₂ amino acid sequence can be found in GenBanK Accession Nos. M12599 and AAA62842 and in Minton et al (Gene 31(1-3), 31-38 (1984)), Minton and Clarke (J. Mol. Appl. Genet. 3(1), 26-35 (1985)); and Chambers et al (Appl. Microbiol. Biotechnol. 29, 572-578 (1998)) and the amino acid sequence is listed in FIG. 1.

In an embodiment the enzyme that has carboxypeptidase G activity may be a derivative of CPG₂ that has carboxypeptidase G activity. By a “derivative” of CPG₂ we include a fragment, variant, modification or fusion of CPG₂, or combinations thereof, which has carboxypeptidase G activity.

The derivatives may be made using protein chemistry techniques for example using partial proteolysis (either exolytically or endolytically), or by de novo synthesis. Alternatively, the derivatives may be made by recombinant DNA technology. Suitable techniques for cloning, manipulation, modification and expression of nucleic acids, and purification of expressed proteins, are well known in the art and are described for example in Sambrook et al (2001) “Molecular Cloning, a Laboratory Manual”, 3^{rd} edition, Sambrook et al (eds), Cold Spring Harbor Laboratory Press, Cold Spring Harbor, N.Y., USA, incorporated herein by reference.

By “fragment” of CPG₂ we mean any portion of the full length enzyme that has carboxypeptidase G activity. Typically, the fragment has at least 30% of the carboxypeptidase G activity of CPG₂. It is more preferred if the fragment has at least 50%, preferably at least 70% and more preferably at least 90% of the activity of CPG₂. Most preferably, the fragment has 100% or more of the carboxypeptidase G activity of CPG₂.

The carboxypeptidase G activity of a derivative of CPG₂ can readily be determined by a person of skill in the art using the enzyme assay described on page 448 of Sherwood et al (1985). The entire disclosure of Sherwood et al (1985) is incorporated herein by reference.

A “variant” of CPG₂ refers to CPG₂ that has been altered by an amino acid insertion, deletion and/or substitution, either conservative or non-conservative, at one or more positions. By “conservative substitutions” is intended combinations such as Gly, Ala; Val, Ile, Leu; Asp, Glu; Asn, Gln; Ser, Thr, Lys, Arg; and Phe, Tyr. Such modifications may be made using the methods of protein engineering and site-directed mutagenesis, as described in Sambrook et al 2001, supra.

For example, it may be advantageous to modify one or more residues of one or both of the active site of the enzyme. Such variants may beneficially alter the specificity or activity of the enzyme. The crystal structure of CPG₂ was published by Rowsell et al (1997) and identifies the active sites of the enzyme. In other embodiments, it may be advantageous not to modify residues in the active sites. Sequence variants, typically outside the active sites, may protect the enzyme from in vivo metabolism or decrease antigenicity. Additionally, it may be advantageous to add one or more Cys residues to allow disulphide bonds to be formed.

Preferably, the variant of CPG₂ has at least 70% sequence identity with SEQ ID No: 1. It is more preferred if variant CPG₂ has at least 80%, preferably at least 85% and more preferably at least 90% sequence identity with SEQ ID No: 1. Most preferably, the variant CPG₂ has 91 or 92 or 93 or 94 or 95 or 96 or 97 or 98 or 99% or more sequence identity with SEQ ID No: 1.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al., (1994) Nucleic Acids Res 22, 4673-80). The parameters used may be as follows:
Fast pairwise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Preferably, the variant of CPG₂, or a fragment of the variant, retains at least 30% of the carboxypeptidase G activity of CPG₂. It is more preferred if variant CPG₂ has at least 50%, preferably at least 70% and more preferably at least 90% of the activity of CPG₂. Most preferably, the variant of CPG₂ has 100% or more of the carboxypeptidase G activity of CPG₂.

Variants of CPG₂ with carboxypeptidase G activity have been described (US patent application no. 2004/0014187).

In an embodiment, the variant of CPG₂ has a substitution at one or more of the Asn residues at positions 222, 264 and 272 which are N-glycosylation sites. Preferably, Asn 222 is substituted with Gln; Asn 264 is substituted with Thr or Ser, most preferably Ser; and Asn 272 is substituted with Gln, independently or in combination. The most preferred combination of substitutions has Gln at positions 222 and 272 and Ser at residue 264. This QSQ motif results in a high catalytic activity and a low Kₘ for MTX (US 2004/0014187).

A “modification” of CPG₂ refers to CPG₂ in which one or more of the amino acid residues has been chemically modified. Such modifications include forming salts with acids or bases, especially physiologically acceptable organic or inorganic acids and bases, forming an ester or amide of a terminal carboxyl group, attaching amino acid protecting groups such as N-t-butoxycarbonyl and glycosylation. Such modifications may protect the enzyme from in vivo metabolism or decrease antigenicity. The CPG₂ may be present as single copies or as multiples, for example tandem repeats.

The invention also includes a fusion of CPG₂, or a fragment or variant thereof which has carboxypeptidase G activity, to another compound. Preferably, the fusion retains at least 30% of the activity of CPG₂. It is more preferred if the fusion has at least 50%, preferably at least 70% and more preferably at least 90% of the activity of CPG₂. Most preferably, the fusion has 100% or more of the carboxypeptidase G activity of CPG₂.

The invention may be used to alleviate symptoms of toxicity caused by the antifolate compound of Formula I in an individual (i.e. palliative use), or may be used to reduce the severity of the toxicity in an individual, or may be used to treat toxicity in an individual, or may be used prophylactically to prevent toxicity in an individual. Thus, by “combating toxicity” we include the meaning of treating, reducing or preventing toxicity caused by the antifolate compound or alleviating the symptoms of it.

The enzyme that has carboxypeptidase G activity typically acts to combat toxicity caused by the antifolate compound of Formula I by rapidly lowering plasma levels of the drug, thereby reducing the duration of exposure of normal tissues to the drug and preventing longer-ter uptake.

Whether or not a particular patient is one who is expected to benefit from treatment may be determined by the physician.

By preventing toxicity we include the meaning of treating a patient at risk of toxicity, for example due to high levels and/or delayed elimination of the antifolate compound of Formula I. Any patient who has been administered the antifolate compound may be considered to be at risk of toxicity caused by it.

In an embodiment, the individual at risk of toxicity may be one who has been administered the antifolate compound and who has not been tested for the presence of a clinical marker of toxicity caused by the antifolate compound.

In another embodiment, the individual at risk of toxicity may be one who has been administered the antifolate compound and has one or more clinical markers of toxicity caused by it.

Thus in an embodiment, the method may comprise the prior step of determining whether the individual who has been administered the antifolate compound of Formula I has a clinical marker of toxicity caused by the antifolate compound.

In an embodiment, the clinical marker of toxicity caused by the antifolate compound of Formula I may be a level of the compound, such as a plasma level, greater than a predetermined level at a given time after administration of the compound. The predetermined plasma level of the antifolate compound indicating toxicity may be 0.1 or 0.2 or 0.3 or 0.4 or 0.5 or 0.6 or 0.7 or 0.8 or 0.9 μmole per litre, or 1 or 2 or 3 or 4 or 5 μmole or more per litre at 24 hours after administration of the antifolate compound, or at 48, or 72 or 96 or 120 hours, or more, after administration of the antifolate compound.

Thus in another embodiment, the method may comprise the prior step of determining the level of the antifolate compound in the individual at a given time after administration of the compound to the individual, such as at 24 or 48, or 72 or 96 or 120 hours, or more, after administration of the antifolate compound.

The invention includes administering an enzyme that has carboxypeptidase G activity to an individual who has been administered an antifolate compound of Formula I as defined above, whether or not the individual has any symptoms of toxicity caused by the compound.

In an embodiment, it may be preferred to administer the enzyme to every individual who has been administered an antifolate compound of Formula I, for example, at a given time after administration of the compound of Formula I.

Thus the invention can be considered to be an in vivo method of cleaving an antifolate compound of Formula I as defined above.

In another embodiment, the individual at risk of toxicity may be one who has been administered the antifolate compound and has one or more clinical symptoms of toxicity caused by it.

Thus in an embodiment, the method may comprise the prior step of determining whether the individual who has been administered the antifolate compound has a clinical symptom of toxicity caused by the antifolate compound.

Symptoms of toxicity for various of the antifolate compounds of Formula I as defined above are well known. For example, toxicities for raltitrexed (Tomudex) include mild mucositis, myelosuppression, neutropaenia, mild anaemia, dehydration, diarrhoea, nausea, astheiria and hepatotoxicity (Tsavaris et al, 2002; Massacesi et al, 2003); toxicites for LY309887 include delayed-onset haematologic, neurologic and mucosal toxicities (Kisliuk, 2003); and toxicities for AG2034 include anaemia, thrombocytopaenia, mucositis, diarrhoea, liver toxicity, fatigue and insomnia (McGuire, 2003).

The individual is typically administered the enzyme that has carboxypeptidase G activity between about 24 and 48 hours after being administered the antifolate compound. Alternatively, the individual may be administered the enzyme between about 12 and 24 hours or between about 48 and 72 hours, or between about 72 and 96 hours, or between about 96 and 120 hours, or more, after being administered the antifolate compound.

The individual may be administered the enzyme that has carboxypeptidase G activity about 6 hours, or about 12 hours, or about 18 hours, or about 24 hours, or about 30 hours, or about 36 hours, or about 42 hours, or about 48 hours, or about 54 hours, or about 60 hours, or about 72 hours, or about 84 hours, or about 96 hours, or about 108 hours, or about 120 hours, or more, after being administered the antifolate compound.

It is appreciated that if the antifolate compound has been administered to the individual in error, the enzyme that has carboxypeptidase G activity is preferably administered as soon as possible once the error is noticed in order to combat toxicity caused by it. Similarly, if the individual has a clinical marker of toxicity caused by the antifolate compound, or a clinical symptom of toxicity caused by the antifolate compound, it may also be preferable to administer the enzyme as soon as possible.

The maximum tolerated dose for Tomudex on a 3-weekly schedule was found to be 3.5 to 4.5 mg/m² in adults and 6 mg/m² in a paediatric population (Clarke et al, 2000). The subsequent administration of an enzyme that has carboxypeptidase G activity followed by degradation of the antifolate compound, can increase the maximum tolerated dose of the antifolate compound thus increasing the efficacy of the drug and minimising any side effects.

The invention thus includes administering to an individual in need thereof, as described herein, a high dose of an antifolate compound of Formula I, such as 2 or 3 or 4 or 5 or 6 or 7 or 8 or 9 or 10 or more times the above doses, and subsequently administering an enzyme that has carboxypeptidase G activity to the individual.

The invention thus includes administering to an adult individual a dose of Tomudex equivalent to about 5 or 6 or 7 or 8 or 9 or 10 or 15 or 20 or 25 or 30 or 40 or 50 mg/m² or more on a 3-weekly schedule, or administering to a child a dose of Tomudex equivalent to 7 or 8 or 9 or 10 or 15 or 20 or 25 or 30 or 40 or 50 mg/m² or more on a 3-weekly schedule, and subsequently administering an enzyme that has carboxypeptidase G activity to the individual.

The enzyme that has carboxypeptidase G activity or a formulation thereof may be administered by any conventional method including parenteral (e.g. subcutaneous or intramuscular) injection. The treatment may consist of a single dose or a plurality of doses over a period of time.

Most preferably, the enzyme that has carboxypeptidase G activity or a formulation thereof is administered intravenously.

In some circumstances the enzyme that has carboxypeptidase G activity or a formulation thereof may be administered intrathecally, typically when the antifolate compound of formula I has been administered intrathecally.

Studies in rhesus monkeys indicate that the half life of CPG₂ in plasma following intravenous administration is between 52 and 58 minutes. Following intrathecal administration to rhesus monkeys, CPG₂'s half life in cerebrospinal fluid has been estimated at between 3.3 and 3.9 hours.

Whilst it is possible for the enzyme that has carboxypeptidase G activity to be administered alone, it is preferable to present it as a pharmaceutical formulation, together with one or more acceptable carriers. The carrier(s) must be “acceptable” in the sense of being compatible with the compound of the invention and not deleterious to the recipients thereof. Typically, the carriers will be water or saline which will be sterile and pyrogen free.

In a preferred embodiment, the enzyme that has carboxypeptidase G activity is stored as a freeze-dried powder ready to be made up as a solution for injection as required. Typically, the contents of a vial of freeze dried enzyme are reconstituted with sterile normal saline (0.9% w/v), immediately before use.

In a preferred embodiment, the formulation of the enzyme that has carboxypeptidase G activity also contains lactose as an inactive ingredient, except for patients with hypersensitivity to lactose.

Typically, the enzyme that has carboxypeptidase G activity is administered to the individual at a dose of about 50 Units per kg body weight (1 unit corresponds to the enzyme activity that cleaves 1 micromole of MTX per minute at 37° C.) intravenously over 5 minutes.

It is appreciated that the enzyme can be administered at lower doses of about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 35, 40 or 45 Units per kg/body weight. It is also appreciated that the enzyme can be administered at higher doses of about 55, 60, 65, 70, 75, 80, 85, 90, 95, 100, 150 or 200 or higher Units per kg/body weight.

The frequency, timing and dosage of administration of the enzyme that has carboxypeptidase G activity may be determined by the physician, using knowledge of the properties of the enzyme, the levels of the antifolate enzyme in the patient, and the degree of any symptoms of toxicity in the patient.

It is appreciated that proteins and peptides may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

An alternative method of protein and peptide delivery is the ReGel injectable system that is thermo-sensitive. Below body temperature, ReGel is an injectable liquid while at body temperature it immediately forms a gel reservoir that slowly erodes and dissolves into known, safe, biodegradable polymers. The active drug is delivered over time as the biopolymers dissolve.

It is further appreciated that the individual may be administered a polynucleotide that encodes the enzyme that has carboxypeptidase G activity, leading to in vivo expression of the enzyme. Suitable vectors and methods are well known to a person of skill in the art.

The individual to be treated may be any individual who would benefit from such treatment. Typically and preferably the individual to be treated is a human. However, the methods of the invention may be used to treat mammals, such as the cows, horses, pigs, sheep, cats and dogs. Thus, the methods have uses in both human and veterinary medicine.

In an embodiment, the method of combating toxicity caused by an antifolate compound of Formula I as defined above further comprises administering a folate pathway rescue agent to the individual.

By “a folate pathway rescue agent” we include the meaning of an agent that can rescue the folate pathway which is blocked by the antifolate compound. The most commonly used folate pathway rescue agent is leucovorin, the calcium salt of 5-formyl tetrahydrofolic acid. Alternative rescue agents may include other salts of 5-formyl tetrahydrofolic acid, and thymidine itself. Typically, if the antifolate compound is an inhibitor of DHFR or of GARFT, the folate pathway rescue agent is leucovorin, while if the antifolate compound is an inhibitor of TS, the folate pathway rescue agent is thymidine.

For example, Tomudex (raltitrexed) is known to be an inhibitor of TS, and LY309987, AG2034 and AG2037 are known to be GARFT inhibitors (McGuire, 2003; Kisliuk, 2003). Thus, the appropriate folate pathway rescue agent can readily be determined.

In an embodiment, the individual is administered the enzyme that has carboxypeptidase G activity prior to the folate pathway rescue agent. Alternatively, the individual may be administered the folate pathway rescue agent prior to the enzyme that has carboxypeptidase G activity. In yet another embodiment, the individual may be administered the folate pathway rescue agent and the enzyme that has carboxypeptidase G activity substantially simultaneously.

The antifolate compounds may be useful in treating a range of medical conditions, and being able to combat toxicity associated with these compounds increases their therapeutic value.

The invention includes a method of treating a disease selected from cancer, RA, MS, psoriasis, extrauterine pregnancy and graft vs. host disease, the method comprising administering antifolate compound of Formula I as defined above, to the individual, and subsequently administering to the individual an enzyme that has carboxypeptidase G activity.

The subsequent administration of the enzyme that has carboxypeptidase G activity is to combat toxicity caused by the antifolate compound as described above.

Thus the invention includes a method of treating a disease selected from cancer, RA, MS, psoriasis, extrauterine pregnancy and graft vs. host disease comprising administering an antifolate compound of Formula I as defined above to the individual, and combating toxicity caused by the antifolate compound as described above.

Cancers which can be treated by the antifolate compounds of Formula I, are well known to a person of skill in the art, some of winch are discussed by McGuire (2003); Kisliuk (2003); and Purcell & Ettinger (2003), as well as in the specific references mentioned below.

The invention includes a method of treating cancer, the method comprising administering Tomudex to the individual, and combating toxicity caused by Tomudex by administering an enzyme that has carboxypeptidase G activity.

The cancer to be treated by administration of Tomudex may be cancer of the breast, ovary, colon/rectum, liver, prostate, pancreas or stomach, as well as non small cell lung cancer (NSCLC), malignant mesothelioma or carcinoma of unknown primary (Clarke et al (2000); Tsavaris et al (2002); Franchi et al (2003); and Massacesi et al (2003)).

The invention includes a method of treating cancer, the method comprising administering LY309887 to the individual, and combating toxicity caused by LY309887 by administering an enzyme that has carboxypeptidase G activity. The cancer to be treated by administration of LY309887 may be cancer of the breast, colon, lung or pancreas (McGuire, 2003).

The invention includes a method of treating cancer, the method comprising administering AG2034 to the individual, and combating toxicity caused by AG2034 by administering an enzyme that has carboxypeptidase G activity. The cancer to be treated by administration of AG2034 may be cancer of the breast, colon or lung, melanoma or lymphoma (McGuire, 2003).

The invention includes a method of treating cancer, the method comprising administering AG2037 to the individual, and combating toxicity caused by AG2037 by administering an enzyme that has carboxypeptidase G activity. The cancer to be treated by administration of AG2037 may be a solid tumour, such as an advanced, metastatic or recurrent solid tumour (McGuire, 2003).

A second aspect of the invention provides the use of an enzyme that has carboxypeptidase G activity in the preparation of a medicament for combating toxicity caused by an antifolate compound of Formula I as defined above in the first aspect of the invention.

Preferences regarding the antifolate compound of Formula I in this and subsequent aspects of the invention are as described above with respect to the first aspect of the invention

The invention includes use of an enzyme that has carboxypeptidase G activity for combating toxicity in an individual who has one of more clinical signs, symptoms or markers of toxicity caused by said compound, as described above.

In an embodiment, the invention includes use of the enzyme that has carboxypeptidase G activity in the preparation of a medicament for combating toxicity caused by an antifolate compound of Formula I as defined above in an individual who is administered a folate pathway rescue agent. The individual may be administered the folate pathway rescue agent prior to the medicament, or the individual may be administered the folate pathway rescue agent after the medicament, or the individual may be administered the folate pathway rescue agent and the medicament substantially simultaneously.

Preferences regarding the folate pathway rescue agent in this and subsequent aspects of the invention are as described above with respect to the first aspect of the invention

A third aspect of the invention provides the use of an antifolate compound of Formula I as defined above in the preparation of a medicament for combating a condition that is combatable by said antifolate compound in an individual who is subsequently administered an enzyme that has carboxypeptidase G activity.

In an embodiment, the individual is also administered a folate pathway rescue agent. The enzyme that has carboxypeptidase G activity may be administered before, after, or substantially simultaneously with the folate pathway rescue agent.

A fourth aspect of the invention provides the use of a folate pathway rescue agent in the preparation of a medicament for combating toxicity caused by an antifolate compound of Formula I as defined above in an individual who is administered an enzyme that has carboxypeptidase G activity. The individual may be administered the enzyme prior to the medicament, or the individual may be administered the enzyme after the medicament, or the individual may be administered the enzyme and the medicament substantially simultaneously.

A fifth aspect of the invention provides the use of an enzyme that has carboxypeptidase G activity and a folate pathway rescue agent in the preparation of a medicament for combating toxicity caused by an antifolate compound of Formula I as defined above.

The invention includes the use as defined above in the second, third, fourth and fifth aspects of the invention for combating toxicity caused by an antifolate compound of Formula I in an individual who is being treated for a disease selected from cancer, RA, MS, psoriasis, extrauterine pregnancy and graft vs. host disease by administration of the antifolate compound, as detailed above.

A sixth aspect of the invention provides the use of an antifolate compound of Formula I as defined above in the preparation of a medicament for treating a condition selected from cancer, RA, MS, psoriasis, extrauterine pregnancy and graft vs. host disease in an individual who is subsequently administered an enzyme that has carboxypeptidase G activity.

In an embodiment, the invention includes the use of Tomudex (raltitrexed) in the preparation of a medicament for treating cancer in an individual who is administered an enzyme that has carboxypeptidase G activity. The invention also includes the use of an enzyme that has carboxypeptidase G activity in the preparation of a medicament for combating toxicity caused by Tomudex. Typically, the individual has been administered the Tomudex to treat cancer.

The cancer to be treated by administration of Tomudex may be cancer of the breast, ovary, colon/rectum, liver, prostate, pancreas or stomach, as well as NSCLC, malignant mesothelioma or carcinoma of unknown primary.

Similarly, cancers that can be treated by administration of a medicament containing LY309887, AG2034 and AG2037 are known in the art, and include those listed above.

The invention thus includes the use of an enzyme that has carboxypeptidase G activity in the preparation of a medicament for complementing the therapy of a disease selected from cancer, RA, MS, psoriasis, extrauterine pregnancy and graft vs. host disease that is being treated by administration of an antifolate compound of Formula I.

A seventh aspect of the invention provides an ex vivo method of cleaving a terminal L-glutamate moiety from a compound of Formula I as defined above, the method comprising contacting the compound with an enzyme that has carboxypeptidase G activity.

An eighth aspect of the invention provides a method of determining the rate and/or extent of cleavage of a compound of Formula I as defined above by an enzyme that has carboxypeptidase G activity, the method comprising:
providing a compound of Formula I,
contacting the compound of Formula I with an enzyme that has carboxypeptidase G activity under conditions such that cleavage of the compound can occur, and
monitoring the rate and/or extent of cleavage of the compound of Formula I over time.

In an embodiment, the providing step comprises providing a known amount or concentration of the compound of Formula I.

In an embodiment, the monitoring step comprises monitoring the amount and/or concentration of the compound of Formula I over time. Additionally or alternatively, the monitoring step comprises monitoring the amount and/or concentration of one or more break-down products of the compound of Formula I over time.

It is appreciated that the method of determining the rate and/or extent of cleavage can be performed ex vivo, or can be performed in vivo.

When the method is performed in vivo it can be used to monitor the level of the compound of Formula I that remains uncleaved after treatment with the enzyme that has carboxypeptidase G activity. Thus the method can be used to monitor the effectiveness of the enzyme that has carboxypeptidase G activity in combating toxicity associated with the compound of Formula I.

The method may further comprise determining whether an additional dose of the enzyme that has carboxypeptidase G activity is required in order to reduce the amount of the compound of Formula I to a predetermined level, typically a level which does not cause toxicity. The amount of enzyme to be administered in the additional dose may also be determined.

The method may therefore also comprise contacting the compound of Formula I with an additional dose of the enzyme that has carboxypeptidase G activity under conditions such that cleavage of the compound of Formula I can occur.

A ninth aspect of the invention provides a therapeutic system (or it may be termed a “kit of parts”) consisting of or comprising an antifolate compound of Formula I as defined above and an enzyme that has carboxypeptidase G activity. Optionally, the therapeutic system may also contain a folate pathway rescue agent.

Preferably, the therapeutic system contains a preferred compound of Formula I as defined above in the first aspect of the invention, most preferably Tomudex. Still preferably, the therapeutic system contains carboxypeptidase G₂, or a derivative thereof that has carboxypeptidase G activity, as defined above in the first aspect of the invention. Preferred folate pathway rescue agents are also as defined in the first aspect of the invention. The therapeutic system or kit of parts may suitably contain both the compound of Formula I and the enzyme that has carboxypeptidase G activity, and optionally the folate pathway rescue agent, packaged and presented in a suitable formulation either for storage or for use. Thus, for example, the erode that has carboxypeptidase G activity may be a freeze-dried powder ready to be reconstituted as a solution for injection, or may already be reconstituted as a solution for injection. Typically, the compound of Formula I and the enzyme are for separate administration in a particular treatment regime, thus they are packaged or formulated separately. The enzyme and the folate pathway rescue agent may be administered together, and thus may be formulated for co-administration.

A preferred therapeutic system or kit as defined above comprises Tomudex, carboxypeptidase G₂ and, optionally, thymidine.

A tenth aspect of the invention provides a method of cleaving a compound comprising a structural fragment of Formula VIII, 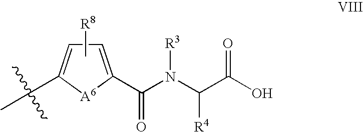
wherein
the wavy line indicates the point of attachment of the structural fragment;
A⁶ represents O or S;
R⁸ represents H or one or two substituents selected from halo, C₁₋₄ alkyl and C₁₋₄ alkoxy;
R³ represents H or C₁₋₄ alkyl;
R⁴ represents —CH₂C(R^{9a})(R^{9b})-D;
R^{9a} and R^{9b} independently represent H or C₁₋₄ allyl, or R^{9a} and R^{9b} together represent ═C(O)R¹⁰;
R¹⁰ represents H or C₁₋₄ alkyl;
D represents C(O)OH, tetrazol-5-yl, (CH₂)₀₋₁—NHR¹¹, or, when R^{9a} and R^{9b} together represent ═C(H)R¹⁰, then D may also represent H, or D represents a structural fragment of Formula IIIa or IIIb, 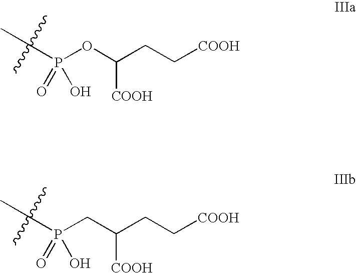
wherein the wavy lines indicate the point of attachment of the structural fragments;
R¹¹ represents H or C(O)R¹²;
R¹² represents H or phenyl substituted by C(O)OH and optionally substituted by one or two further substituents selected from halo, C₁₋₄ alkyl and C₁₋₄ alkoxy; and
alkyl, alkenyl and alkynyl groups, as well as the alkyl part of alkoxy groups, may be substituted by one or more halo atoms;
or a pharmaceutically acceptable salt and/or solvate thereof,
the method comprising contacting the compound comprising the structural fragment of Formula VIII with an enzyme that has carboxypeptidase G activity.

Preferably, A⁶ represents S.

Preferences for the enzyme that has carboxypeptidase G activity are the same as for the first aspect of the invention as described above.

In an embodiment, the method may be performed ex vivo, such as in vitro.

In an alternative embodiment, the method may be performed in vivo.

Typically, the compound comprising the structural fragment of Formula VIII is an antifolate compound.

In an embodiment when the antifolate compound comprising a structural fragment of Formula VIII is administered to an individual in the course of medical treatment, or otherwise, the invention provides a method of combating toxicity caused by the antifolate compound, the method comprising administering to the individual an enzyme that has carboxypeptidase G activity.

Thus the invention includes the use of an enzyme that has carboxypeptidase G activity in the preparation of a medicament for combating toxicity caused by an antifolate compound comprising a structural fragment of Formula VIII.

In the context of medical uses of this tenth aspect of the invention when the compound comprising the structural fragment of Formula VIII is an antifolate compound, preferences for the use of folate pathway rescue agents, pharmaceutical formulations, timings and levels of administration, patients and diseases to be treated, and so on are the same as for the first aspect of the invention as described above.

All of the documents referred to herein are incorporated herein, in their entirety, by reference.

The listing or discussion of a prior-published document in this specification should not necessarily be taken as an acknowledgement that the document is part of the state of the art or is common general knowledge.

The invention will now be described in more detail by reference to the following figures and Examples.

FIG. 1 is the amino acid sequence of CPG₂ (SEQ ID No: 1).

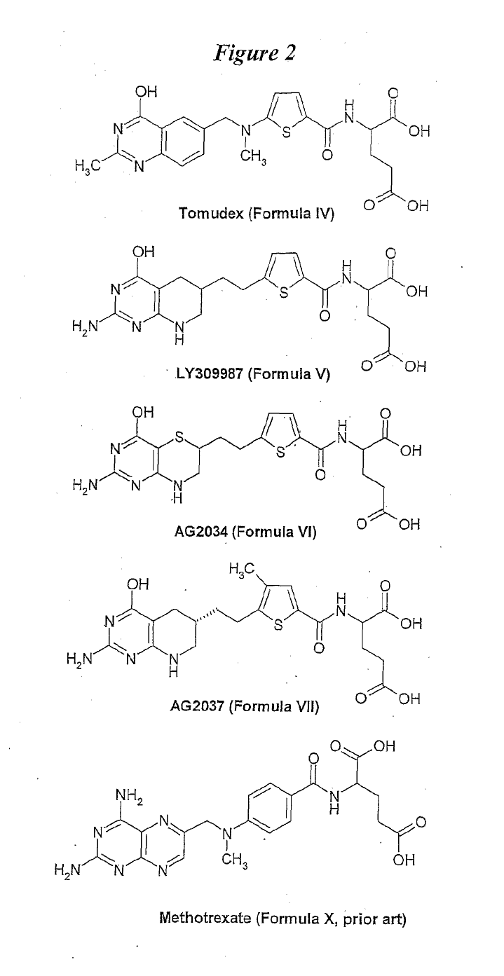

FIG. 2 shows the chemical structure of five substrates of CPG₂. Tomudex (Formula IV), LY309887 (Formula V), AG2034 (Formula VI), AG2037 (Formula VII) and Methotrexate (Formula X, prior art).

### EXAMPLE 1

### Identification of New Substrates for CPG₂

A: Tomudex (Formula IV)

Tomudex is a thymidilate synthase inhibitor (Astra Zeneca). Tomudex was contacted with CPG₂ in vitro and was found to a substrate of CPG₂ as described below.

Deglutamylation of Tomudex by CPG₂ was measured spectrophotometrically. A stock solution of 10 mM Tomudex in 100 mM Tris-HCl pH7.3 was prepared and used to prepare a series of dilutions from 0-100 μM in 100 mM Tris-HCl pH 7.3. 990 μl of each dilution was placed in a quartz cuvette and pre-warmed to 37° C. 10 μl of enzyme solution was added to initiate the reaction and the progress of the reaction was followed by measuring the rate of change in absorbance at 356 nm. The change in extinction coefficient of Tomudex at 356 nm following complete conversion of a 100 μM solution by an excess of CPG₂ was measured in order to calculate the change in extinction coefficient due to the complete conversion of 100 nmol of Tomudex. With this data, measured rates can then be converted to values of μmol/min (Vₘₐₓ), and Kₘ and k_{cat} determined.

Measurement of the rate of reaction at a range of substrate concentrations, from 1-100 μM allows the Kₘ and k_{cat} for the enzyme to be determined using appropriate computer software such as “Enzfitter” (Biosoft; Cambridge, UK). Results of the spectrophotometric assay showed that Tomudex is a substrate for CPG₂ having a Kₘ of 7.8 μM and a K_{cat} of 24/s.

B: LY309887 (Formula V)

LY309887 is a GARFT inhibitor (Eli Lilly). LY309887 is contacted with CPG₂ in vitro and is found to be a substrate of CPG₂.

Deglutamylation of LY309887 by CPG₂ is measured spectrophotometrically. A stock solution of 10 mM LY309887 in 100 mM Tris-HCl pH7.3 is prepared and used to prepare a series of dilutions from 0-100 μM in 100 mM Tris-HCl pH 7.3. 990 μl of each dilution is placed in a quartz cuvette and pre-warned to 37° C. 10 μl of enzyme solution is added to initiate the reaction and the progress of the reaction is followed by measuring the rate of change in absorbance at a suitable wavelength. The change in extinction coefficient of LY309887 following complete conversion of a 100 μM solution by an excess of CPG₂ is measured in order to calculate the change in extinction coefficient due to the complete conversion of 100 nmol of LY309887. With this data, measured rates are converted to values of μmol/min. Measurement of the rate of reaction at a range of substrate concentrations, from 1-100 μM allows the Kₘ and k_{cat} for the enzyme to be determined using Enzfitter computer software.

C: AG2034 (Formula VI)

The GARFT inhibitor AG2034 is contacted with CPG₂ in vitro and is found to be a substrate of CPG₂.

Deglutamylation of AG2034 by CPG₂ is measured spectrophotometrically. A stock solution of 10 mM AG2034 in 100 mM Tris-HCl pH7.3 is prepared and used to prepare a series of dilutions from 0-100 μM in 100 mM Tris-HCl pH 7.3. 990 μl of each dilution is placed in a quartz cuvette and pre-warmed to 37° C. 10 μl of enzyme solution is added to initiate the reaction and the progress of the reaction is followed by measuring the rate of change in absorbance at a suitable wavelength. The change in extinction coefficient of AG2034 following complete conversion of a 100 μM solution by an excess of CPG₂ is measured in order to calculate the change in extinction coefficient due to the complete conversion of 100 nmol of AG2034. With this data, measured rates are converted to values of μmol/min. Measurement of the rate of reaction at a range of substrate concentrations, from 1-100 μM allows the Kₘ and k_{cat} for the enzyme to be determined using Enzfitter computer software.

D:AG2037 (Formula VII)

The GARFT inhibitor AG2037 is contacted with CPG₂ in vitro and is found to be a substrate of CPG₂.

Cleavage of AG2037 by CPG₂ is measured spectrophotometrically. A stock solution of 10 mM AG2037 in 100 mM Tris-HCl pH7.3 is prepared and used to prepare a series of dilutions from 0-100 μM in 100 mM Tris-HCl pH 7.3. 990 μl of each dilution is placed in a quartz cuvette and pre-warmed to 37° C. 10 μl of enzyme solution is added to initiate the reaction and the progress of the reaction is followed by measuring the rate of change in absorbance at a suitable wavelength. The change in extinction coefficient of AG2037 following complete conversion of a 100 μM solution by an excess of CPG₂ is measured in order to calculate the change in extinction coefficient due to the complete conversion of 100 nmol of AG2037. With this data, measured rates are converted to values of μmol/min. Measurement of the rate of reaction at a range of substrate concentrations, from 1-100 μM allows the Kₘ and k_{cat} for the enzyme to be determined using Enzfitter computer software.

### EXAMPLE 2

### Rescue of Tomudex Toxicity by Administration of CPG₂

A patient who has been administered Tomudex and has toxic plasma levels of Tomudex is administered a dose of 50 Units per kg body weight of CPG₂ by intravenous injection over a period of about 5 minutes. The patient's plasma concentration of Tomudex is reduced to non-toxic levels.

### EXAMPLE 3

### Rescue of Tomudex Toxicity by Administration of CPG₂ and Thymidine

A patient who has been administered Tomudex and has toxic plasma levels of Tomudex is administered a dose of 50 Units per kg body weight of CPG₂ by intravenous injection over a period of about 5 minutes and is also administered thymidine. The thymidine rescues the cellular toxicity associated with the Tomudex, while the CPG₂ drastically reduces the plasma concentration of Tomudex to non-toxic levels.

### LIST OF REFERENCES

Adamson, P C et al (1991) “Rescue of experimental intrathecal methotrexate overdose with carboxypeptidase-G2.” J Clin Oncol 9: 670-674.
Bleyer, W A (1918) “The Clinical Pharmacology of Methotrexate. New applications for an old drug.” Cancer 41: 36-51.
Chabner, B et al (1972) “Enzymatic cleavage of methotrexate provides a method for prevention of drug toxicity.” Nature 239: 395-397.
Clarke S J et al (2000) “Clinical and preclinical pharmacokinetics of raltitrexed.” Clin Pharmacokinet. 39(6): 429-43.
Condit, P et al (1969) “Renal toxicity of methotrexate.” Cancer 23: 126-131.
Dowell, R I et al (1996) “New mustard prodrugs for antibody-directed enzyme prodrug therapy: alternatives to the amide link.” J. Med. Chem. 39: 1100-1105.
Franchi F et al, (2003) “Favorable toxicity profile of raltitrexed in elderly patients treated for colorectal cancer: a case series.” Gerontology 49(5): 324-7
Goldman, I (1975) “Membrane transport of methotrexate and other folate compounds: relevance to rescue protocols.” Cancer Chemo Rep 6: 63-72.
Jolivet, J et al (1983) “The pharmacology and clinical use of methotrexate.” N Engl J Med 309: 1094-1104.
Kalchatgi, K and Bertino J (1981) “Folate-degrading enzymes: a review with special emphasis on Carboxypeptidase G.” In: Enzymes as drugs J Holcenberg and J Roberts, eds, Wiley, New York, pp 77-102.
Kintzel, P E (2001) “Anticancer drug-induced kidney disorders.” Drug Saf 24: 19-38.
Kisliuk R L (2003) “Deaza analogs of folic acid as antitumor agents.” Current Pharmaceutical Design 9(31): 2615-2625.
Massacesi C et al, (2003) “Raltitrexed-induced hepatotoxicity: multivariate analysis of predictive factors.” Anticancer Drugs 14(7): 533-41.
McCullough, J et al (1971) “Purification and properties of Carboxypeptidase G1.” J Biol Chem 246: 7207-7213.
McGuire JJ (2003) “Anticancer antifolates: current status and future directions” Current Pharmaceutical Design 9(31): 2593-2613.
Minton, N P et al (1983) “Molecular cloning of the Pseudomonas Carboxypeptidase G2 gene and its expression in Escherichia coli and Pseudomnonasputida.” J Bacteriol 156: 1222-1227.
Mohty, M et al (2000) “Carboxypeptidase G2 rescue in delayed methotrexate elimination in renal failure.” Leuk Lymphoma 37: 441-443.
Pinedo, H et al (1976) “The reversal of methotrexate cytotoxicity to mouse bone marrow cells by leucovorin and nucleosides.” Cancer Res 36: 4418-4424.
Purcell, W T & Ettinger D S (2003) “Novel antifolate drugs” Current Oncology Reports 5(2): 114-125.
Rowsell, S et al (1997) “Crystal structure of carboxypeptidase G2, a bacterial enzyme with applications in cancer therapy.” Structure 5(3): 337-47.
Sherwood, R F et al (1985) “Purification and properties of Carboxypeptidase G2 from Pseudomonas sp. strain RS-16. Use of a novel triazine dye affinity method.” Eur J Biochem 148: 447-453.
Springer, C J et al (1995) “Optimization of alkylating agent prodrugs derived from phenol and aniline mustards: a new clinical candidate prodrug (ZD2767) for antibody-directed enzyme prodrug therapy.” J. Med. Chem. 38: 5051-65.
Tsavaris, N et al (2002) “Raltitrexed (Tomudex) administration in patients with relapsed metastatic colorectal cancer after weekly irinotecan/5-Fluorouracil/Leucovorin chemotherapy.” BMC Cancer. 2(1): 2. Epub 2002 Jan. 30.
Von Poblozki, A et al (2000) “Carboxypeptidase-G2 rescue in a woman with methotrexate-induced renal failure.” Med Klin 95: 457-460.
Widemann, B C et al (2000) “Pharmacokinetics and metabolism of the methotrexate metabolite 2,4-diamino-Nlo-methylpteroic acid.” J Pharmacol Expel Therapy 294: 894-901.

1. A method of combating toxicity caused by an antifolate compound of Formula I, 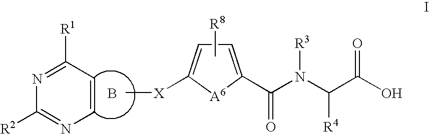
wherein
R¹ represents NH₂, OH or CH₃;
R² represents NH₂ or C₁₋₄ alkyl;
the group B represents a structural fragment of Formula Ia, Ib, Ic, Id or Ie, 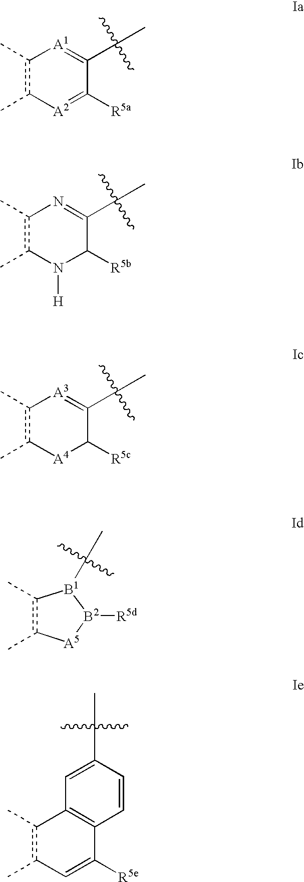
in which groups the dashed lines indicate the point of ring fusion with the pyrimidinyl ring and the wavy lines indicate the point of attachment of the structural fragments to the group X;
R^{5a} to R^{5e} independently represent H or C₁₋₄ alkyl;
A¹ represents C(R^{6a}) or N;
A² represents CH or N;
A³ represents C(H)R^{6b}, NR^{6c} or S;
A⁴ and A⁵ independently represent CH₂, NH, O or S;
the group B¹-B² represents CH—CH or C═C;
R^{6a} to R^{6c} independently represent H or C₁₋₄ alkyl, or R^{6c} represents C(O)R^{6d}, or
R^{6c}, together with R^{7b} represents C₁₋₂ n-alkylene;
R^{6d} represents H or C₁₋₄ alkyl;
X represents —CH₂C(H)R^{7a}— or —CH₂NR^{7b}—(in which latter two groups the CH₂ moiety is attached to the fused, pyrimidine-based heterocyclic group);
R^{7a} and R^{7b} independently represent H, C₁₋₆ alkyl, C₃₋₆ alkenyl or C₃₋₆ alkynyl, or
R^{7b}, together with R^{6c} represents C₁₋₂ n-alkylene;
A⁶ represents O or S;
R⁸ represents H or one or two substituents selected from halo, C₁₋₄ alkyl and C₁₋₄ alkoxy;
R³ represents H or C₁₋₄ alkyl;
R⁴ represents —CH₂C(R^{9a})(R^{9b})-D;
R^{9a} and R^{9b} independently represent H or C₁₋₄ aryl, or R^{9a} and R^{9b} together represent —C(H)R¹⁰,
R¹⁰ represents H or C₁₋₄ alkyl;
D represents C(O)OH, tetrazol-5-yl, (CH₂)₀₋₁—NHR¹¹, or, when R^{9a} and R^{9b} together represent ═C(H)R¹⁰, then D may also represent H, or D represents a structural fragment of Formula IIIa or IIIb, 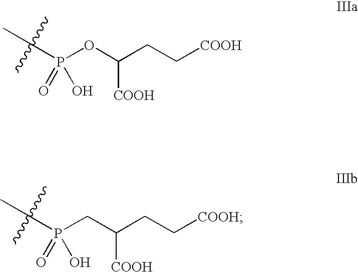
wherein the wavy lines indicate the point of attachment of the structural fragments;
R¹¹ represents H or C(O)R¹²;
R¹² represents H or phenyl substituted by C(O)OH and optionally substituted by one or two further substituents selected from halo, C₁₋₄ alkyl and C₁₋₄ alkoxy; and
alkyl, alkenyl and alynyl groups, as well as the alkyl part of alkoxy groups, may be substituted by one or more halo atoms;
or a pharmaceutically acceptable salt and/or solvate thereof,
in an individual who has been administered said compound, the method comprising administering to the individual an enzyme that has carboxypeptidase G activity.

2. A method according to claim 1 wherein the antifolate compound of Formula I is Tomudex (Formula IV), LY309887 (Formula V), AG2034 (Formula VI) or AG2037 (Formula VII).

3. A method according to claim 1 or 2 wherein the individual is administered the enzyme that has carboxypeptidase G activity between about 24 and 48 hours after being administered the antifolate compound.

4. A method according to any of claims 1 to 3 wherein the individual has one of more clinical markers of toxicity caused by the antifolate compound.

5. A method according to claim 4 wherein the clinical marker of toxicity caused by the antifolate compound is a plasma level of the compound greater than a predetermined level indicating toxicity at a given time after administration of the compound.

6. A method according to claim 5 wherein the predetermined blood plasma level of the antifolate compound indicating toxicity is 1 μM at 24 hours after administration of the compound.

7. A method according to claim 5 or 6 further comprising the prior step of determining the plasma level of the antifolate compound in the individual at a given time after administration of the compound.

8. A method according to any of claims 1 to 7 wherein the individual has one or more clinical symptoms of toxicity caused by the antifolate compound.

9. A method according to claim 8 wherein the clinical symptom of toxicity caused by the antifolate compound is selected from anaemia, anorexia, asthenia, dehydration, diarrhoea, fatigue, fever, hepatotoxicity, hyperbilirubinaemia, leukopaenia, mucositis, myelosuppression, nausea, neutropaenia, rash, reversible transaminitis, stomatitis, thrombocytopaenia and vomiting.

10. A method according to claim 8 or 9 further comprising the prior step of determining the presence of the one or more clinical symptoms of toxicity caused by the antifolate compound in the individual.

11. A method according to any of claims 1 to 10 and further comprising administering a folate pathway rescue agent to the individual.

12. A method according to claim 11 wherein the antifolate compound of Formula I is an inhibitor of dihydrofolate reductase (DHFR) or of glycinamide ribonucleotide formyltransferase (GARFT), and the folate pathway rescue agent is leucovorin calcium.

13. A method according to claim 12 wherein the antifolate compound is LY309887, AG2034, or AG2037.

14. A method according to claim 11 wherein the antifolate compound of Formula I is an inhibitor of thymidylate synthase (TS), and the folate pathway rescue agent is thymidine.

15. A method according to claim 14 wherein the antifolate compound is Tomudex.

16. A method according to any of claims 11 to 15 wherein the individual is administered the enzyme that has carboxypeptidase G activity prior to the folate pathway rescue agent.

17. A method according to any of claims 11 to 15 wherein the individual is administered the folate pathway rescue agent prior to the enzyme that has carboxypeptidase G activity.

18. A method according to any of claims 11 to 15 wherein the individual is administered the folate pathway rescue agent and the enzyme that has carboxypeptidase G activity substantially simultaneously.

19. A method according to any of claims 1 to 18 wherein the individual is administered the enzyme that has carboxypeptidase G activity at a dose of about 50 Units per kg body weight.

20. A method of treating a disease selected from cancer, rheumatoid arthritis (RA), multiple sclerosis (MS), psoriasis, extrauterine pregnancy and graft vs. host disease comprising administering an antifolate compound of Formula I to the individual, and subsequently administering to the individual an enzyme that has carboxypeptidase G activity.

21. A method according to claim 20 wherein the antifolate compound of Formula I is Tomudex and the cancer to be treated is cancer of the breast, ovary, colon/rectum, liver, prostate, pancreas or stomach, or non small cell lung cancer (NSCLC), malignant mesothelioma or carcinoma of unknown primary.

22. A method according to claim 20 wherein the antifolate compound is LY309887 and the cancer to be treated is cancer of the breast, colon, lung or pancreas.

23. A method according to claim 20 wherein the antifolate compound is AG2034 and the cancer to be treated is breast cancer, colon cancer, lung cancer, melanoma or lymphoma.

24. A method according to claim 20 wherein the antifolate compound is AG2037 and the cancer to be treated is a solid tumour, such as an advanced, metastatic or recurrent solid tumour.

25. Use of an enzyme that has carboxypeptidase G activity in the preparation of a medicament for combating toxicity caused by an antifolate compound of Formula I as defined in claim 1 or claim 2.

26. Use according to claim 25 for combating toxicity in an individual who has one of more clinical markers of toxicity caused by the antifolate compound.

27. Use according to claim 26 wherein the clinical marker of toxicity is a plasma level of the antifolate compound greater than a predetermined level indicating toxicity at a given time after administration of the compound.

28. Use according to claim 27 wherein the predetermined plasma level of the antifolate compound indicating toxicity is 1 μM at 24 hours after administration of the compound.

29. Use according to any of claims 25 to 28 for combating toxicity in an individual who has one of more clinical symptoms of toxicity caused by the antifolate compound.

30. Use according to claim 29 wherein the clinical symptom of toxicity caused by the antifolate compound is selected from anaemia, anorexia, asthenia, dehydration, diarrhoea, fatigue, fever, hepatotoxicity, hyperbilirubinaemia, leukopaenia, mucositis, myelosuppression, nausea, neutropaenia, rash, reversible transaminitis, stomatitis, thrombocytopaenia and vomiting.

31. Use of an antifolate compound of Formula I as defined in claim 1 or claim 2 in the preparation of a medicament for combating a disease combatable by said antifolate compound in an individual who is subsequently administered an enzyme that has carboxypeptidase G activity.

32. Use according to any of claims 25 to 31 for combating toxicity in an individual who is administered a folate pathway rescue agent.

33. Use according to claim 32 wherein the individual is administered the folate pathway rescue agent prior to the enzyme that has carboxypeptidase G activity.

34. Use according to claim 32 wherein the individual is administered the folate pathway rescue agent after the enzyme that has carboxypeptidase G activity.

35. Use according to claim 32 wherein the individual is administered the folate pathway rescue agent and the enzyme that has carboxypeptidase G activity substantially simultaneously.

36. Use of a folate pathway rescue agent in the preparation of a medicament for combating toxicity caused by an antifolate compound of Formula I as defined in claim 1 or claim 2 in an individual who is administered an enzyme that has carboxypeptidase G activity.

37. Use of an enzyme that has carboxypeptidase G activity and a folate pathway rescue agent in the preparation of a medicament for combating toxicity caused by an antifolate compound of Formula I as defined in claim 1 or claim 2.

38. Use according to any of claims 32 to 37 wherein the antifolate compound is an inhibitor of DHFR or GARFT, and the folate pathway rescue agent is leucovorin.

39. Use according to claim 38 wherein the antifolate compound is LY309887, AG2034, or AG2037.

40. Use according to any of claims 32 to 37 wherein the antifolate compound of Formula I is an inhibitor of TS, and the folate pathway rescue agent is thymidine.

41. Use according to claim 40 wherein the antifolate compound of Formula I is Tomudex.

42. Use according to any of claims 25 to 41 wherein the enzyme that has carboxypeptidase G activity is at a dose of about 50 Units per kg body weight.

43. Use according to any of claims 25 to 42 for combating toxicity caused by an antifolate compound of Formula I in an individual who is being treated for a disease selected from cancer, RA, MS, psoriasis, extrauterine pregnancy and graft vs. host disease by administration of the antifolate compound.

44. Use of an antifolate compound of Formula I as defined in claim 1 or claim 2 in the preparation of a medicament for treating a condition selected from cancer, RA, MS, psoriasis, extrauterine pregnancy and graft vs. host disease in an individual who is subsequently administered an enzyme that has carboxypeptidase G activity.

45. Use of an enzyme that has carboxypeptidase G activity in the preparation of a medicament for complementing the therapy of a disease selected from cancer, RA, MS, psoriasis, extrauterine pregnancy and graft vs. host disease that is being treated by administration of an antifolate compound of Formula I.

46. Use according to any of claims 43 to 45 wherein the antifolate compound of Formula I and the cancer to be treated are as defined in any of claims 21-24.

47. A therapeutic system comprising an antifolate compound of Formula I as defined above in claim 1 or 2, and an enzyme that has carboxypeptidase G activity.

48. A therapeutic system according to claim 47 further comprising a folate pathway rescue agent.

49. An ex vivo method of cleaving a terminal L-glutamate moiety from a compound of Formula I as defined in claim 1 or claim 2, the method comprising contacting the compound with an enzyme that has carboxypeptidase G activity.

50. A method of determining the rate and/or extent of cleavage of a compound of Formula I as defined in claim 1 or claim 2 by an enzyme that has carboxypeptidase G activity, the method comprising:
providing the compound of Formula I,
contacting the compound of Formula I with an enzyme that has carboxypeptidase G activity under conditions such that cleavage of the compound can occur, and
monitoring the rate and/or extent of cleavage of the compound of Formula I over time.

51. A method according to claim 50 wherein the monitoring step comprises monitoring the amount and/or concentration of the compound of Formula I.

52. A method according to claim 50 or 51 wherein the monitoring step comprises monitoring the amount and/or concentration of one or more break-down products of the compound of Formula I.

53. A method according to any of claims 50 to 52 which is performed ex vivo.

54. A method according to any of claims 50 to 52 which is performed in vivo.

55. A method according to claim 54 further comprising determining whether an additional dose of the enzyme that has carboxypeptidase G activity is required in order reduce the amount of the compound of Formula I to a predetermined level.

56. A method according to claim 54 or 55 further comprising contacting the compound of Formula I with an additional dose of the enzyme that has carboxypeptidase G activity under conditions such that cleavage of the compound can occur.

57. A method of cleaving a compound comprising a structural fragment of Formula VIII, 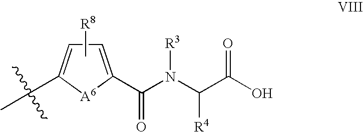
wherein
the wavy line indicates the point of attachment of the structural fragment;
A⁶ represents O or S;
R⁸ represents H or one or two substituents selected from halo, C₁₋₄ alkyl and C₁₋₄ alkoxy;
R³ represents H or C₁₋₄ alkyl;
R⁴ represents —CH₂C(R^{9a})(R^{9b})-D;
R^{9a} and R^{9b} independently represent H or C₁₋₄ alkyl, or R^{9a} and R^{9b} together represent ═C(H)R¹⁰;
R¹⁰ represents X or C₁₋₄ alkyl;
D represents C(O)OH, tetrazol-5-yl, (CH₂)₀₋₁—NHR¹¹, or, when R^{9a} and R^{9b} together represent —C(H)R¹⁰, then D may also represent H, or D represents a structural fragment of Formula IIIa or IIIb, 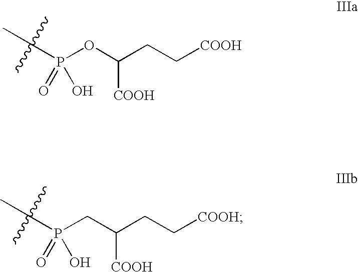
wherein the wavy lines indicate the point of attachment of the structural fragments;
R¹¹ represents H or C(O)R¹²;
R¹² represents H or phenyl substituted by C(O)OH and optionally substituted by one or two further substituents selected from halo, C₁₋₄ alkyl and C₁₋₄ alkoxy; and
ally, alkenyl and alkynyl groups, as well as the alkyl part of alkoxy groups, may be substituted by one or more halo atoms;
or a pharmaceutically acceptable salt and/or solvate thereof,
the method comprising contacting the compound comprising the structural fragment of Formula VIII with an enzyme that has carboxypeptidase G activity.

58. A method according to claim 57 that is performed ex vivo.

59. A method according to claim 57 that is performed in vivo.

60. A method according to claim 57 wherein the compound comprising the structural fragment of Formula VIII is an antifolate compound.

61. A method according to claim 60 for combating toxicity caused by the antifolate compound in an individual who has been administered the said antifolate compound in the course of medical treatment, or otherwise, the method comprising administering to the individual an enzyme that has carboxypeptidase G activity.

62. Use of an enzyme that has carboxypeptidase G activity in the preparation of a medicament for combating toxicity caused by an antifolate compound of Formula VIII as defined in claim 57.

63. A method according to any of claims 1 to 24 or 49 to 61, or a use according to any of claims 25 to 46 or 62, or a therapeutic system according to claim 47 or 48, wherein the enzyme that has carboxypeptidase G activity is carboxypeptidase G₂, or a derivative thereof which has carboxypeptidase G activity.


SEQUENCE LISTING (1 sequence)

SEQ ID NO: 1 - protein, 415 aa, Variovorax paradoxus
MRPSIHRTAIAAVLATAFVAGTALAQKRDNVLFQAATDEQPAVIKTLEKLVNIETGTGDAEGIAAAGNFLEAELKNLGFTVTRSKSAGLVVGDNIVGKIKGRGGKNLLLMSHMDTVYLKGILAKAPFRVEGDKAYGPGIADDKGGNAVILHTLKLLKEYGVRDYGTITVLFNTDEEKGSFGSRDLIQEEAKLADYVLSFEPTSAGDEKLSLGTSGIAYVQVNITGKASHAGAAPELGVNALVEASDLVLRTMNIDDKAKNLRFNWTIAKAGNVSNIIPASATLNADVRYARNEDFDAAMKTLEERAQQKKLPEADVKVIVTRGRPAFNAGEGGKKLVDKAVAYYKEAGGTLGVEERTGGGTDAAYAALSGKPVIESLGLPGFGYHSDKAEYVDISAIPRRLYMAARLIMDLGAGK